(19) United States
(12) Patent Application Publication
(10) Pub. No.: US 2008/0045890 A1
(43) Pub. Date: Feb. 21, 2008

(54) METHODS AND SYSTEMS FOR ABLATING TISSUE

(75) Inventors: Kirk Patrick Seward, Dublin, CA (US); Juan Granada, Pearland, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Mercator MedSystems, Inc., San Leandro, CA (US)

(21) Appl. No.: 11/567,539

(22) Filed: Dec. 6, 2006

Related U.S. Application Data
(60) Provisional application No. 60/751,372, filed on Dec. 16, 2005.

(51) Int. Cl. A61M 31/00

(52) U.S. Cl. 604/93.01

(57) ABSTRACT

Methods and systems for treating patients requiring tissue ablation for volumetric tissue reduction rely on the injection of ethanol and other tissue-ablating agents into the perivascular space surrounding body lumens, particularly blood vessels or vessels of the alimentary canal, reproductive system and urinary tract. Injection of tissue-ablating agents is intended treat conditions such as hypertrophic cardiomyopathy, benign and malignant tumors, benign prostatic hyperplasia, and uterine fibroids, for example. Injection may be achieved using intravascular catheters which advance needles radially outward from a body vessel lumen or by transmyocardial injection from an epicardial or endocardial surface of the heart.

### CROSS-REFERENCES TO RELATED APPLICATIONS

The present application claims the benefit of provisional U.S. Application No. 60/751,372 (Attorney Docket No. 021621-002300US), filed Dec. 16, 2005, the full disclosure of which is incorporated herein by reference.

### BACKGROUND OF THE INVENTION

### 1. Field of the Invention

The present invention relates generally to medical devices, systems, and methods. More particularly, the present invention relates to methods and systems for ablating tissue by the direct injection of tissue-ablating agents. Even more particularly, the present invention relates to methods and systems for ablating tissue by the direct perivascular or periventricular injection of tissue-ablating agents.

Hyperproliferative and hypertrophic disorders involve the proliferation of cells or thickening of tissues in the body and can result from injury, cancer, congenital disease, and other medical trauma. Scar tissue, tumors, and thickened walls of the ventricles of the heart are each examples of these disorders.

An exemplary disease resulting from a hypertrophic disorder is hypertrophic cardiomyopathy (HCM), also referred to as idiopathic hypertrophic subaortic stenosis (IHSS), asymmetrical septal hypertrophy (ASH), or hypertrophic obstructive cardiomyopathy (HOCM). This disease results in a thickening of the interventricular septum of the heart and can lead to decreased ability for the heart to pump blood and obstruction of the ventricular outflow. Hypertrophic cardiomyopathy has a prevalence rate of 1 in 500 in the U.S. population. Obstruction of ventricular outflow occurs in 25% of patients with HCM and can lead to sudden cardiac death. Those patients are typically treated with drugs like beta blockers, calcium channel blockers, anti-arrhythmics, and diuretics. The 5% of patients that do not respond to medications require surgical or interventional therapy to remove part of the septal wall or ablate part of the septum with pure ethanol.

Current ablation therapy for HOCM involves placement of a balloon angioplasty catheter into the first septal artery, inflation of the balloon to prevent retrograde flow back into the left anterior descending artery (LAD) and infusion of 0.5 to 5 ml of desiccated ethanol. Five minutes later, the balloon is deflated and removed from the body. The infusion of alcohol leads to occlusion of the septal artery and infarction of the myocardium of the septum. Consequent thinning of the septal wall leads to an immediate relief of high ventricular outflow pressure gradients. However, the occlusion of the septal artery can also cut off blood flow to the atrioventricular node (A-V Node) and can result in arrhythmia requiring temporary or permanent implantation of a pacemaker. Other complications include alcohol leaking back into the LAD and causing occlusion and further infarction. Predominant concerns about alcohol septal ablation via septal artery infusion include the long-term risk for arrhythmia-related events including sudden cardiac death.

Other diseases have been similarly treated with alcohol ablation, including hepatic tumors and benign prostatic hyperplasia.

For these reasons, it would be desirable to provide improved methods and systems for delivering tissue-ablating agents such as alcohol directly to tissue. It would be particularly desirable if tissues could be accessed with percutaneous cardiovascular catheters in order to reduce surgical morbidity and mortality risk. Such methods and systems will preferably be catheter-based and permit introduction of the alcohol and other tissue-ablating agents into cardiac and other tissue near the coronary and peripheral vasculature, including both arteries and veins, and should further provide delivery of such agents to precisely controlled locations within or adjacent to the target tissues, and should still further provide for the direct delivery of such agents into tissue without dilution in the systemic circulation. Further preferably, the methods and system should allow for the injection of the alcohol and other agents in the tissue surrounding the coronary and peripheral vasculature in regions which permit the direct visualization of distribution of the agents to desired regions of tissue in amounts and at levels sufficient to provide the desired therapeutic benefits. At least some of these objectives will be met by the inventions described hereinafter.

### BRIEF SUMMARY OF THE INVENTION

The present invention provides improved methods and systems for ablating tissue in patients for whom tissue ablation is recommended to decrease tissue thickness or volume. Methods and systems will be particularly suitable for treating patients who suffer from hypertrophic cardiomyopathy (HCM), benign prostatic hyperplasia (BPH) or solid tumors such as hepatomas. Methods and systems of the present invention rely on the direct delivery of tissue-ablating agents, particularly alcohols, and more particularly ethanol, to tissue, particularly tissue for which volumetric reduction is sought, usually employing a catheter for injection of the drugs beyond the endothelium of an artery or vein into the perivascular space beyond the outside of the external elastic lamina so that the agent is able to permeate into perivascular tissue requiring ablation, but also sometimes employing a catheter for injection of the drugs directly into cardiac tissue via an approach through one of the chambers, particularly the ventricles, of the heart.

Current methods utilized for alcohol ablation are described in detail in Li et al. (2003) Int J. Card. 91:93-96, Maron et al. (2003) J Am Coll Cardiol. 42:13-16, Chang et al. (2004) Circulation. 109:824-827, van Dockum et al. (2004) J Am Coll Cardiol. 43(1):27-34, Goya et al. (1999) J. Urol. 162:383-386, Seggewiss et al. (1998) J Am Coll Cardiol. 31(2):252-258, Knight et al. (1997) Circulation 95:2075-2081, and Gietzen et al. (2004) Heart 90:638-644. Description of the blood supply to the atrioventricular node is described in Abuin and Nieponice (1998) Tex Heart Inst J 24:113-117.

A particular advantage of the present invention is the ability to deliver the tissue-ablating agents directly into tissue where ablation is desired. It is presently believed that the current intraluminal infusion of alcohol into the septal artery ablates the arterial tissue as a primary action and the occlusion of the artery leads to subsequent tissue ischemia, necrosis, and volumetric reduction. The ablation of the septal artery may also lead to ablation of the A-V Node, disrupting the electrical circuitry of the heart and requiring the implantation of a permanent pacemaker. It is believed that direct injection of ethanol mixed with contrast medium to the outside of the septal artery will lead to ablation of the target myocardial tissue with less damage to the heart's electrical functions, thus requiring fewer pacemaker implantations to ameliorate side effects of the current intraluminal ablation procedure. The contrast medium provides the operating physician with a positive feedback of presence of injectate and thus extent of tissue ablation.

Another particular advantage of the present invention is the ability to deliver the tissue-ablating agent while visualizing the dispersion of the agent with a contrast medium that can be viewed by X-ray fluoroscopy, ultrasonic guidance, nuclear magnetic resonance, or the like. Typically, the contrast medium will be a radio-opaque contrast that can be visualized by X-ray imaging. An exemplary concentration of the contrast in the solution is 10% to 90%, with the remainder of the solution as the tissue-ablating agent. Typically, the tissue-ablating agent will be ethanol, either in a 100% solution or diluted in saline or water for injection.

The current procedure typically utilized for alcohol septal ablation involves monitoring by angiogram the outflow rate of the septal artery and then infusing 0.5 to 5 ml of pure ethanol after subjectively judging the length of time that the ethanol will remain in the artery. It is believed that the variability among patients and physicians results in inconsistency in ablated septal mass and thus difficulty in procedure requiring highly specialized physicians.

It is believed that the ability to monitor the dispersion or diffusion of agents during injection will correspond with the amount of tissue ablated. Successful tissue ablation procedures in patients with HCM have resulted from an ablation of approximately 20% of the septum, or 3% to 10% of the left ventricular mass. It is believed that the ability to visualize the volume diffusion and correlate that to septal ablation will enable far more accuracy in the septal ablation procedure.

The methods and systems of the present invention preferably utilize injection from an endovascular or endocardial device in order to deliver the tissue-ablating agents to the perivascular space or myocardial tissue as defined above. Use of intravascular delivery is particularly preferred with those patients who are not undergoing procedures which would result in either open chest, intercostal, thoracoscopic or other direct access to the epicardial surface. Once such direct access is provided, however, the methods of the present invention may be performed by injection transmyocardially from an epicardial surface to the target perivascular space surrounding the blood vessel. Accurate positioning of the needle may be achieved using, for example, transesophogeal imaging, flouroscopic imaging, or the like.

In particular, the preferred intravascular injection methods of the present invention comprise injecting a tissue-ablating agent into the adventitial and perivascular tissues by advancing a needle from a lumen of a blood vessel, or in some cases, an alimentary vessel such as the urethra, to the target location beyond the vessel wall. The tissue-ablating agent is then delivered through the needle to the target tissues. The needle is at least into the perivascular space beyond the outside of the endothelium of the blood vessel or beyond the wall of an alimentary vessel, and usually is advanced into the tissue that has been targeted for ablation surrounding the blood vessel.

The tissue-ablating agents will be injected under conditions and in an amount sufficient to permeate the perivascular tissue around of the vessel and into the surrounding over length of at least about 1 cm, and usually at least about 2 cm or greater. Thus, the needle may be advanced in a radial direction to a depth in the tissue surrounding the vessel equal to at least 10% of the mean luminal diameter of the blood vessel at the site of direct injection, more typically being in the range from 10% to 150%, usually from 10% to 50% of the mean luminal diameter.

Systems according to the present invention for treating a patient suffering from a disease requiring ablation of tissue, particularly hypertrophic cardiomyopathy, comprise an amount of a tissue-ablating agent, particularly a mixture of ethanol, saline or water for injection, and a contrast medium, sufficient to ablate a desirable volume of tissue and an intravascular catheter having a needle for injecting the drug into a location beyond the endothelium of the blood vessel as described above.

### BRIEF DESCRIPTION OF THE DRAWINGS

FIG. 1A is a schematic, perspective view of an intravascular injection catheter suitable for use in the methods and systems of the present invention.

FIG. 1B is a cross-sectional view along line 1B-1B of FIG. 1A.

FIG. 1C is a cross-sectional view along line IC-IC of FIG. 1A.

FIG. 2A is a schematic, perspective view of the catheter of FIGS. 1A-1C shown with the injection needle deployed.

FIG. 2B is a cross-sectional view along line 2B-2B of FIG. 2A.

FIG. 3 is a schematic, perspective view of the intravascular catheter of FIGS. 1A-1C injecting tissue-ablation agent into an adventitial space surrounding a coronary blood vessel in accordance with the methods of the present invention.

FIG. 4 is a schematic, perspective view of another embodiment of an intravascular injection catheter useful in the methods of the present invention.

FIG. 5 is a schematic, perspective view of still another embodiment of an intravascular injection catheter useful in the methods of the present invention, as inserted into a patient's vasculature.

FIGS. 6A and 6B are schematic views of other embodiments of an intravascular injection catheter useful in the methods of the present invention (in an unactuated condition) including multiple needles.

FIG. 7 is a schematic view of yet another embodiment of an intravascular injection catheter useful in the methods of the present invention (in an unactuated condition).

FIG. 8 is a perspective view of a needle injection catheter useful in the methods and systems of the present invention.

FIG. 9 is a cross-sectional view of the catheter FIG. 8 shown with the injection needle in a retracted configuration.

FIG. 10 is a cross-sectional view similar to FIG. 9, shown with the injection needle laterally advanced into luminal tissue for the delivery of tissue-ablation agent according to the present invention.

FIG. 11 is a cross-sectional view of a heart, shown with a trans-endocardial, or intraventricular, needle-injection catheter advanced into the septal wall for the delivery of tissue-ablation agent according to the present invention.

### DETAILED DESCRIPTION OF THE INVENTION

The present invention provides methods and systems for ablating tissues, typically in patients with hyperproliferative or hypertrophic diseases. In particular, these patients will have been diagnosed or otherwise determined to be suffering from obstructive hypertrophic cardiomyopathy. In other cases, however, patients who have hyperproliferative tumors, benign prostatic hyperplasia, or other disorders that may require ablation of tissues may also be candidates for receiving treatment according to the present invention in order to reduce the size or presence of certain tissues in the body.

The present invention will preferably utilize devices and methods for intravascular approach and transvascular or transventricular injection of the ablating agent. The following description provides several representative embodiments of microneedles and macroneedles suitable for the delivery of the agents into a perivascular space or adventitial tissue or directly into myocardial tissue by trans-endocardial injection catheter. The perivascular space is the potential space between the outer surface and the endothelium or “vascular wall” of either an artery or vein. The microneedle is usually inserted substantially normal to the wall of a vessel (artery or vein) to eliminate as much trauma to the patient as possible. Until the microneedle is at the site of an injection, it is positioned out of the way so that it does not scrape against arterial or venous walls with its tip. Specifically, the microneedle remains enclosed in the walls of an actuator or sheath attached to a catheter so that it will not injure the patient during intervention or the physician during handling. When the injection site is reached, movement of the actuator along the vessel terminated, and the actuator is operated to cause the microneedle to be thrust outwardly, substantially perpendicular to the central axis of a vessel, for instance, in which the catheter has been inserted.

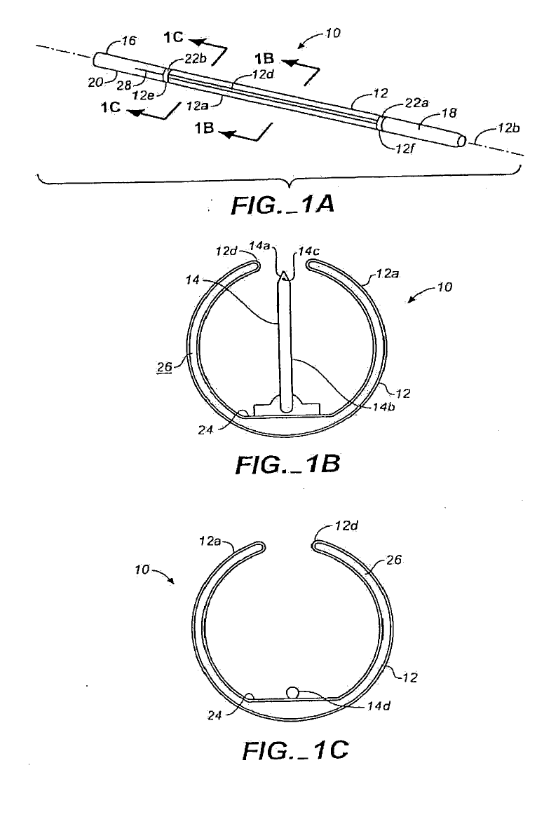

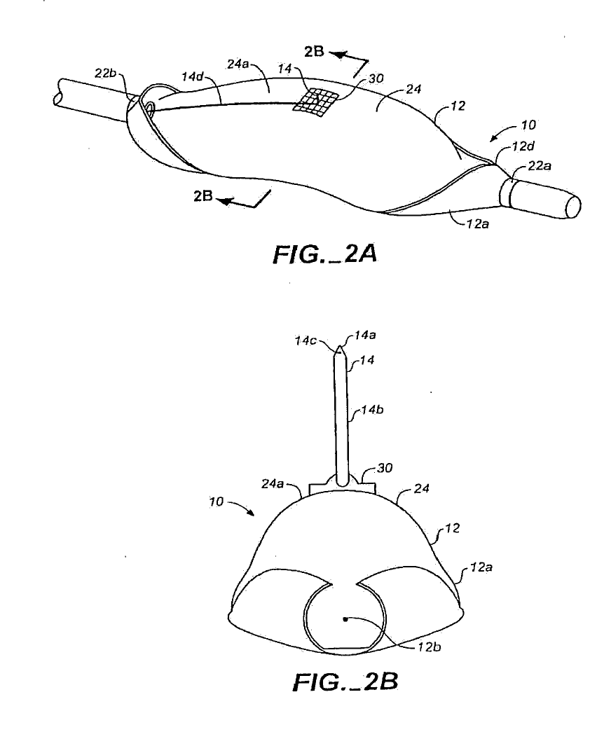

As shown in FIGS. 1A-2B, a microfabricated intravascular catheter 10 includes an actuator 12 having an actuator body 12a and central longitudinal axis 12b. The actuator body more or less forms a C-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a blood vessel by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b may be located at the distal and proximal ends, respectively, of the actuator or may be excluded. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings form a rigid substantially “C”-shaped structure at each end of the actuator. The catheter may be joined to the retaining rings by, for example, a butt-weld, an ultra sonic weld, integral polymer encapsulation or an adhesive such as an epoxy.

The actuator body further comprises a central, expandable section 24 located between retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 psi upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflont© or other inert plastics. The activating fluid may be a saline solution or a radio-opaque dye.

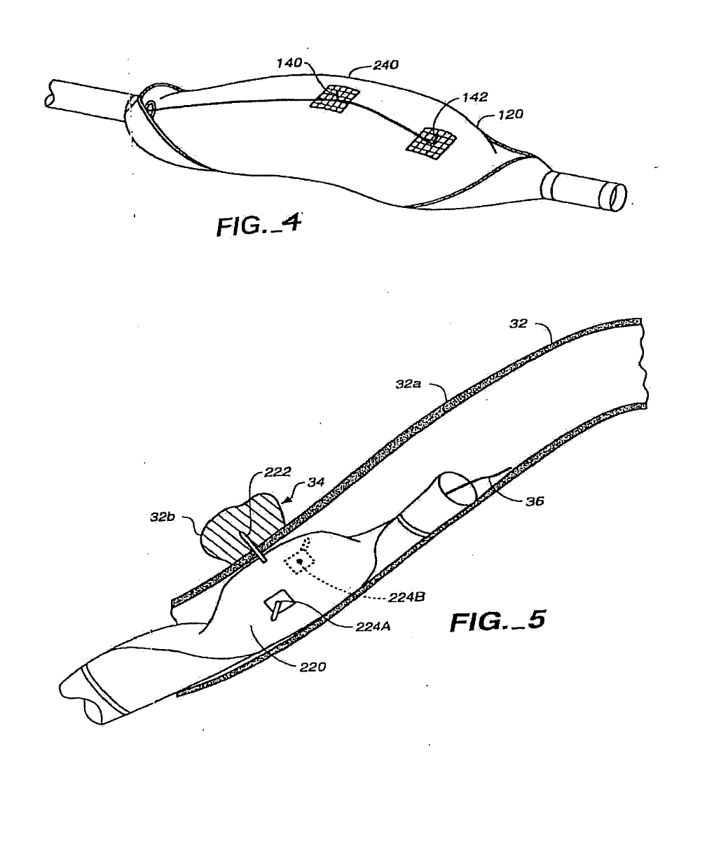

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate. Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon.

The microneedle includes a sharp tip 14a and a shaft 14b. The microneedle tip can provide an insertion edge or point. The shaft 14b can be hollow and the tip can have an outlet port 14c, permitting the injection of a pharmaceutical or tissue-ablation agent into a patient. The microneedle, however, does not need to be hollow, as it may be configured like a neural probe to accomplish other tasks.

As shown, the microneedle extends approximately perpendicularly from surface 24a. Thus, as described, the microneedle will move substantially perpendicularly to an axis of a vessel or artery into which has been inserted, to allow direct puncture or breach of vascular walls.

The microneedle further includes a pharmaceutical or tissue-ablation agent supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass.

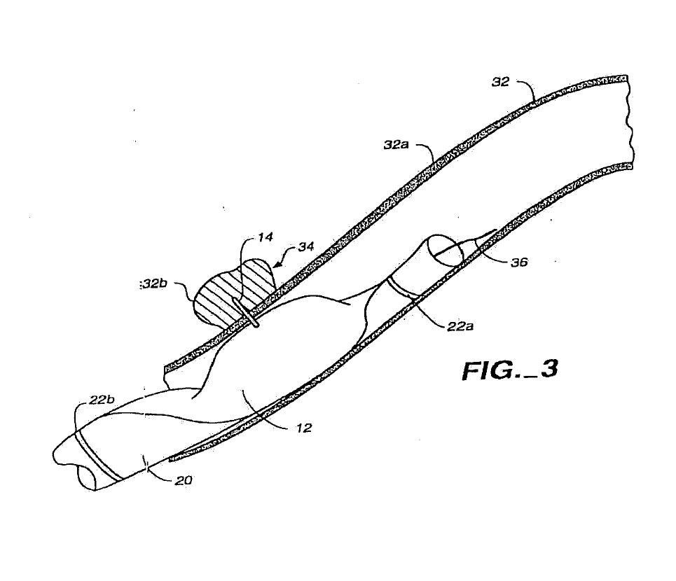

The catheter 20, in use, is inserted through an artery or vein and moved within a patient's vasculature, for instance, a vein 32, until a specific, targeted region 34 is reaches (see FIG. 3). The targeted region 34 may be the site of tissue damage or more usually will be adjacent the sites typically being within 100 mm or less to allow migration of the therapeutic agents. As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire.

During maneuvering of the catheter 20, well-known methods of fluoroscopy or magnetic resonance imaging (MRI) can be used to image the catheter and assist in positioning the actuator 12 and the microneedle 14 at the target region. As the catheter is guided inside the patient's body, the microneedle remains unfurled or held inside the actuator body so that no trauma is caused to the vascular walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and two seconds for the microneedle to move from its furled state to its unfurled state.

The ends of the actuator at the retaining rings 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels so quickly and with such force that it can enter perivascular tissue 32b as well as vascular tissue. Additionally, since the actuator is “parked” or stopped prior to actuation, more precise placement and control over penetration of the vascular wall are obtained.

After actuation of the microneedle and delivery of the cells to the target region via the microneedle, the activating fluid is exhausted from the open area 26 of the actuator, causing the expandable section 24 to return to its original, furled state. This also causes the microneedle to be withdrawn from the vascular wall. The microneedle, being withdrawn, is once again sheathed by the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include an intravascular ultrasonic sensor (IVUS) for locating vessel walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 um, while the tube's and shaft's exterior cross-sectional dimension may be between about 100 and 500 μm. The overall length of the actuator body may be between about 5 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 100-500 μm. The diameter of the delivery tube for the activating fluid may be about 100 μm. The catheter size may be between 1.5 and 15 French (Fr).

Variations of the invention include a multiple-buckling actuator with a single supply tube for the activating fluid. The multiple-buckling actuator includes multiple needles that can be inserted into or through a vessel wall for providing injection at different locations or times.

For instance, as shown in FIG. 4, the actuator 120 includes microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t.sub.1, and pressure p.sub.1, and the microneedle 142 moving at time t.sub.2 and P.sub.2, with t.sub.1, and p.sub.1, being less than t.sub.2 and P.sub.2, respectively.

This sort of staged buckling can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a vessel wall. A needle 224B (as shown in phantom) could alternatively be arranged to move at angle of about 180° to the needle 224A.

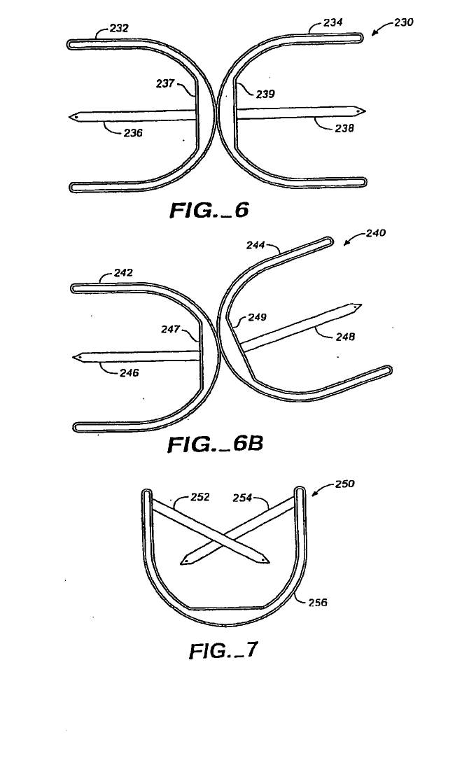

Moreover, as shown in FIG. 6A, in another embodiment, an actuator 230 comprises actuator bodies 232 and 234 including needles 236 and 238, respectively, that move approximately horizontally at angle of about 180° to each other. Also, as shown in FIG. 7B, an actuator 240 comprises actuator bodies 242 and 244 including needles 242 and 244, respectively, that are configured to move at some angle relative to each other than 90° or 180°. The central expandable section of the actuator 230 is provided by central expandable sections 237 and 239 of the actuator bodies 232 and 234, respectively. Similarly, the central expandable section of the actuator 240 is provided by central expandable sections 247 and 249 of the actuator bodies 242 and 244, respectively.

Additionally, as shown in FIG. 7, an actuator 250 may be constructed that includes multiple needles 252 and 254 that move in different directions when the actuator is caused to change from the unactuated to the actuated condition. The needles 252 and 254, upon activation, do not move in a substantially perpendicular direction relative to the longitudinal axis of the actuator body 256.

The above catheter designs and variations thereon, are described in U.S. Pat. Nos. 6,547,803 and 6,860,867, the full disclosures of which are incorporated herein by reference. Co-pending application Ser. Nos. 10/350,314 and 10/691,119, assigned to the assignee of the present application, describes the ability of substances delivered by direct injection into the adventitial and pericardial tissues of the heart to rapidly and evenly distribute within the heart tissues, even to locations remote from the site of injection. The full disclosure of those co-pending applications are also incorporated herein by reference. An alternative needle catheter design suitable for delivering the tissue-ablation agents of the present invention will be described below. That particular catheter design is described and claimed in co-pending application Ser. No. 10/393,700 (Attorney Docket No. 021621-001500 U.S.), filed on Mar. 19, 2003, the full disclosure of which is incorporated herein by reference.

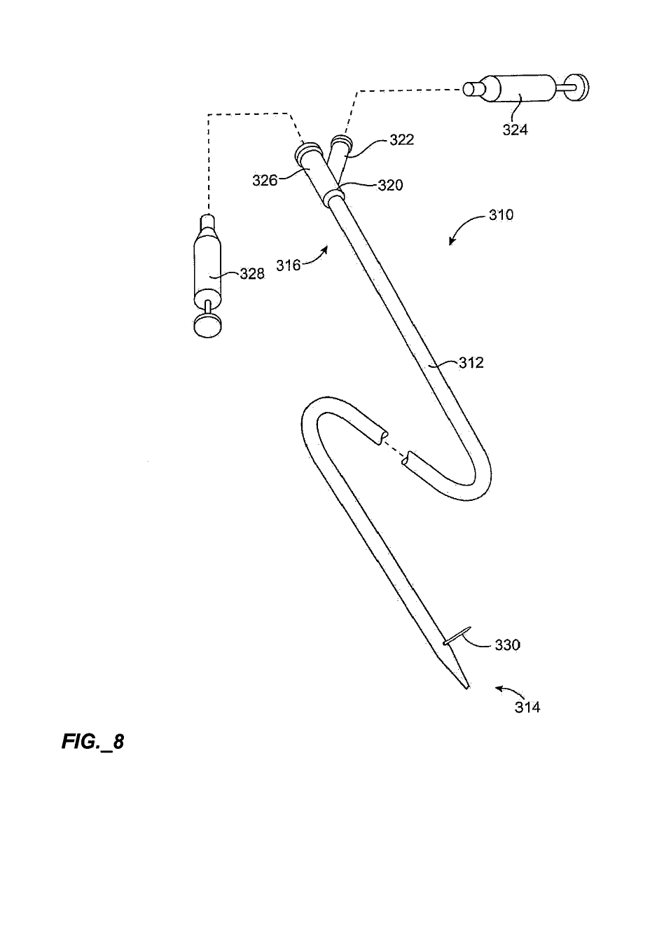

Referring now to FIG. 8, a needle injection catheter 310 constructed in accordance with the principles of the present invention comprises a catheter body 312 having a distal end 314 and a proximal 316. Usually, a guide wire lumen 313 will be provided in a distal nose 352 of the catheter, although over-the-wire and embodiments which do not require guide wire placement will also be within the scope of the present invention. A two-port hub 320 is attached to the proximal end 316 of the catheter body 312 and includes a first port 322 for delivery of a hydraulic fluid, e.g., using a syringe 324, and a second port 326 for delivering the pharmaceutical agent, e.g., using a syringe 328. A reciprocatable, deflectable needle 330 is mounted near the distal end of the catheter body 312 and is shown in its laterally advanced configuration in FIG. 8.

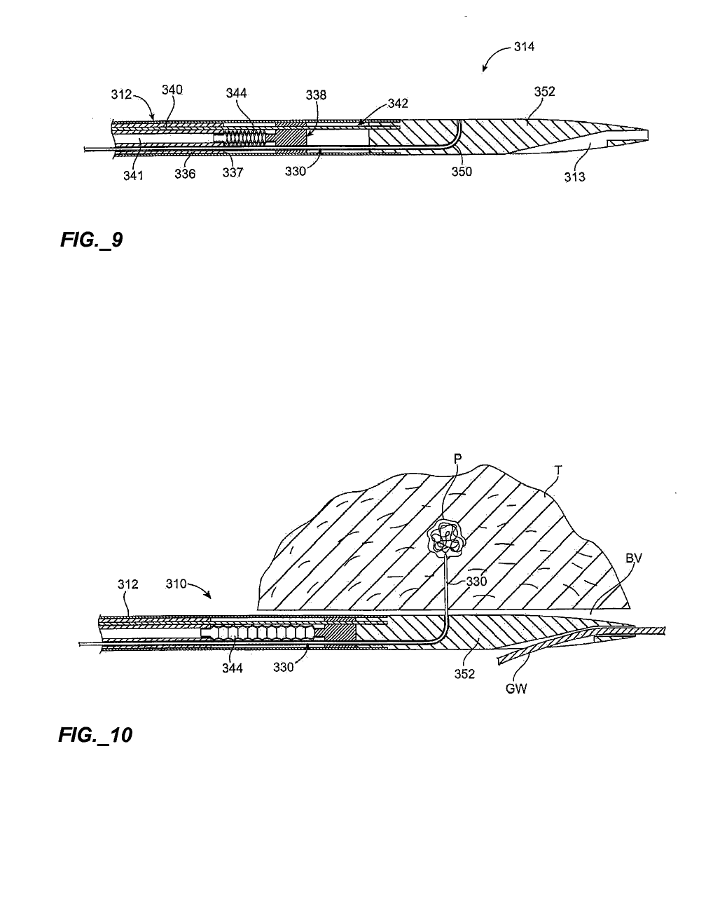

Referring now to FIG. 9, the proximal end 314 of the catheter body 312 has a main lumen 336 which holds the needle 330, a reciprocatable piston 338, and a hydraulic fluid delivery tube 340. The piston 338 is mounted to slide over a rail 342 and is fixedly attached to the needle 330. Thus, by delivering a pressurized hydraulic fluid through a lumen 341 tube 340 into a bellows structure 344, the piston 338 may be advanced axially toward the distal tip in order to cause the needle to pass through a deflection path 350 formed in a catheter nose 352.

As can be seen in FIG. 10, the catheter 310 may be positioned in a coronary blood vessel BV, over a guide wire GW in a conventional manner. Distal advancement of the piston 338 causes the needle 330 to advance into luminal tissue T adjacent to the catheter when it is present in the blood vessel. The tissue-ablation agent may then be introduced through the port 326 using syringe 328 in order to introduce a plume P of tissue-ablation agent in the cardiac tissue, as illustrated in FIG. 10. The plume P will be within or adjacent to the region of tissue damage as described above.

The needle 330 may extend the entire length of the catheter body 312 or, more usually, will extend only partially in tissue-ablation agent delivery lumen 337 in the tube 340. A proximal end of the needle can form a sliding seal with the lumen 337 to permit pressurized delivery of the tissue-ablation agent through the needle.

The needle 330 will be composed of an elastic material, typically an elastic or super elastic metal, typically being nitinol or other super elastic metal. Alternatively, the needle 330 could be formed from a non-elastically deformable or malleable metal which is shaped as it passes through a deflection path. The use of non-elastically deformable metals, however, is less preferred since such metals will generally not retain their straightened configuration after they pass through the deflection path.

The bellows structure 344 may be made by depositing by parylene or another conformal polymer layer onto a mandrel and then dissolving the mandrel from within the polymer shell structure. Alternatively, the bellows 344 could be made from an elastomeric material to form a balloon structure. In a still further alternative, a spring structure can be utilized in, on, or over the bellows in order to drive the bellows to a closed position in the absence of pressurized hydraulic fluid therein.

After the tissue-ablation agent is delivered through the needle 330, as shown in FIG. 10, the needle is retracted and the catheter either repositioned for further agent delivery or withdrawn. In some embodiments, the needle will be retracted simply by aspirating the hydraulic fluid from the bellows 344. In other embodiments, needle retraction may be assisted by a return spring, e.g., locked between a distal face of the piston 338 and a proximal wall of the distal tip 352 (not shown) and/or by a pull wire attached to the piston and running through lumen 341.

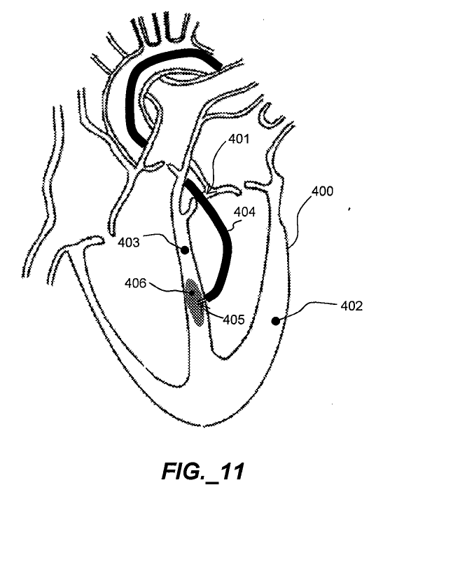

Additionally, as shown in FIG. 11, a catheter is advanced through the aortic valve 401 of a heart 400. In the case of hypertrophic cardiomyopathy, the left ventricular wall 402 and septum 403 are abnormally thick. In advanced cases of this disease, the septal wall may require ablation to prevent it from occluding the outflow of blood through the aortic valve. A catheter 404 is advanced to the septal wall of the left ventricle and a needle 405 is advanced into the septal wall for the delivery of tissue-ablation agent. The agent 406 diffuses upon injection and is visualized with contrast medium to determine the volume of tissue ablated.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for treating a patient, said method comprising delivering a tissue-ablating agent to perivascular tissue in a patients' body.

2. A method as in claim 1, wherein delivering comprises injecting the tissue-ablating agent through the endothelium of a vessel.

3. A method as in claim 2, wherein the vessel is an artery.

4. A method as in claim 3, wherein the artery is a septal artery.

5. A method as in claim 2, wherein the vessel is a vein.

6. A method as in claim 2, wherein the vessel is a urethra.

7. A method as in claim 1, wherein delivering comprises injecting the tissue-ablating agent transmyocardially into heart tissue.

8. A method as in claim 1, wherein the tissue-ablating agent is ethanol or a composition of ethanol and a contrast medium or a composition of ethanol, contrast medium and a diluent.

9. A method as in claim 2, wherein injecting comprises advancing a needle from a lumen of a blood vessel to the location beyond the endothelium and infusing the agent through the needle.

10. A method as in claim 9, wherein the needle is advanced into a perivascular space beyond the outside of the endothelium.

11. A method as in claim 10, wherein the needle is advanced into the adventitia surrounding the vessel.

12. A method as in claim 10, wherein the needle is advanced into the myocardium surrounding the vessel.

13. A method as in claim 2, wherein the tissue-ablating agent is injected in an amount sufficient to permeate a total tissue volume of at least 0.5 cm³.

14. A method as in claim 2, wherein the needle is advanced in a radial direction to a depth in the perivascular tissue equal to at least 10% of the mean luminal diameter at the vessel location.

15. A method as in claim 14, wherein the depth is a distance in the range from 10% to 150% of the mean luminal diameter.

16. A method as in claim 1, wherein the tissue is cardiac tissue which is abnormally thickened due to hypertrophic cardiomyopathy.

17. A method as in claim 1, wherein the tissue is prostate tissue affected by benign prostatic hyperplasia.

18. A method as in claim 1, wherein the tissue is proximate a tumor or multiple tumors.

19. A method as in claim 1, wherein the tissue is a tumor.

20. A method as in claim 1, wherein the tissue is a uterine fibroid.

21. A method for treating a patient suffering from a obstructive hypertrophic cardiomyopathy, said method comprising:
advancing a needle from a lumen of a blood vessel to the location beyond the endothelium of the blood vessel in a target cardiac tissue region;
and injecting ethanol or a composition of ethanol and a contrast medium or a composition of ethanol, contrast medium and a diluent through the needle into tissue at a location beyond the endothelium of the vessel.

22. A method as in claim 21, wherein the blood vessel is a coronary artery.

23. A method as in claim 21, wherein the blood vessel is a coronary vein.

24. A method as in claim 21, wherein the target cardiac tissue region is the cardiac septum.

25. A method as in claim 21, wherein the needle is advanced into a perivascular space beyond the outside of the endothelium.

26. A method as in claim 21, wherein the needle is advanced into the adventitia and/or periadventitial tissue surrounding the blood vessel.

27. A method as in claim 21, wherein the ethanol or a composition of ethanol and a contrast medium or a composition of ethanol, contrast medium and a diluent is injected in an amount sufficient to permeate a total tissue volume of at least 0.5 cm³.

28. A method as in claim 21, wherein the needle is advanced in a radial direction to a depth in the adventitia equal to at least 10% of the mean luminal diameter at the blood vessel location.

29. A method as in claim 28, wherein the depth is a distance in the range from 10% to 150% of the mean luminal diameter.

30. A method as in claim 21, wherein the cardiac tissue is abnormally thick due to hypertrophic cardiomyopathy.

31. A system for ablating tissue, said system comprising:
an amount of a tissue-ablating agent selected to ablate tissue when delivered to a location beyond the endothelium of a blood vessel; and
an intravascular catheter having a needle for injecting the tissue-ablating agent into a location beyond the endothelium of a blood vessel.

32. A system as in claim 31, wherein the tissue-ablating agent comprises ethanol or a composition of ethanol and a contrast medium or a composition of ethanol, contrast medium and a diluent.

33. A method for treating a patient suffering from a obstructive hypertrophic cardiomyopathy, said method comprising:
advancing a needle from inside a chamber of the heart into a target cardiac tissue region; and
injecting ethanol or a composition of ethanol and a contrast medium or a composition of ethanol, contrast medium and a diluent through the needle into the tissue.

34. A method as in claim 33, wherein the target cardiac tissue region is the cardiac septum.

35. A method as in claim 33, wherein the ethanol or a composition of ethanol and a contrast medium or a composition of ethanol, contrast medium and a diluent is injected in an amount sufficient to permeate a total tissue volume of at least 0.5 cm³.

36. A method as in claim 33, wherein the cardiac tissue is abnormally thick due to hypertrophic cardiomyopathy.

37. A system for ablating tissue, said system comprising:
an amount of a tissue-ablating agent selected to ablate tissue when delivered into cardiac tissue; and
an intravascular catheter having a needle for injecting the tissue-ablating agent that can be advanced from inside a chamber of the heart into a target cardiac tissue region.

38. A system as in claim 37, wherein the tissue-ablating agent comprises ethanol or a composition of ethanol and a contrast medium or a composition of ethanol, contrast medium and a diluent.

39. A method for treating a patient suffering from benign prostatic hyperplasia, said method comprising:
advancing a needle from within the urinary tract to the location beyond the wall of the urinary vessel in a target prostate tissue region; and
injecting ethanol or a composition of ethanol and a contrast medium or a composition of ethanol, contrast medium and a diluent through the needle into tissue at a location beyond the endothelium of the vessel.

40. A system for ablating tissue, said system comprising:
an amount of a tissue-ablating agent selected to ablate tissue when delivered into prostate or benign prostatic hyperplastic tissue; and
an intra-urethral catheter having a needle for injecting the tissue-ablating agent that can be advanced from inside the urethra into a target tissue region.

41. A system as in claim 40, wherein the tissue-ablating agent comprises ethanol or a composition of ethanol and a contrast medium or a composition of ethanol, contrast medium and a diluent.

42. A method for treating a patient suffering from benign or malignant tumor(s), said method comprising:
advancing a needle from within a body lumen to a location beyond the wall of the vessel surrounding the body lumen in a target tissue region proximate the tumor or tumors; and
injecting ethanol or a composition of ethanol and a contrast medium or a composition of ethanol, contrast medium and a diluent through the needle into tissue at a location beyond the wall of the vessel.

43. A system for ablating tissue, said system comprising:
an amount of a tissue-ablating agent selected to ablate tumor tissue when delivered into or proximate to the tumor; and
an intravascular catheter having a needle for injecting the tissue-ablating agent that can be advanced from inside a body lumen into the target tissue region.

44. A system as in claim 43, wherein the tissue-ablating agent comprises ethanol or a composition of ethanol and a contrast medium or a composition of ethanol, contrast medium and a diluent.
